(19) United States
(12) Patent Application Publication
(10) Pub. No.: US 2005/0137492 A1
(43) Pub. Date: Jun. 23, 2005

(54) Detection of acoustic nerve tumors

(76) Inventors: Jos J. Eggermont, Calgary (CA); Joseph C. Dort, Calgary (CA)

Correspondence Address: THOMPSON LAMBERT;SUITE 703D, CRYSTAL PARK TWO, 2121 CRYSTAL DRIVE, ARLINGTON, VA, 22202, US

(21) Appl. No.: 10/780,615

(22) Filed: Feb. 19, 2004

(30) Foreign Application Priority Data
Dec. 23, 2003 (CA) 2,451,929

(51) Int. Cl. A61B 5/04; A61B 5/00

(52) U.S. Cl. 600/544; 600/559

(57) ABSTRACT

A method of predicting a tumor of the auditory nerve based on the latency of the auditory brainstem response (ABR) in the presence of a tumor. The ABR is masked using white noise and derived bands are calculated. The wideband (WB) response is also recorded. The derived bands are transformed and summed to form a SUM, and the transform of the WB is also taken. The ratio SUM/WB is taken, and the result is compared to normal ratios to predict the presence of a tumor.

### BACKGROUND OF THE INVENTION

A tumor of the auditory nerve affects the ABR (auditory brainstem response) in individuals. This fact has been used as a screening method for MRI (magnetic resonance imaging) testing. The test commonly used involves stimulating the cochlea by a “click”, and comparing the response to the national average for males or females. While the test was quite accurate for large tumors, it was less accurate for smaller tumors. Improvements were made on the test by windowing the acoustic response into “derived bands”, or bands which displayed the ABR in a particular frequency range.

It is known in the art how a tumor of the auditory nerve affects the individual derived bands. The derived bands tend to be present in acoustic tumor ABRs but they are differentially shifted to longer latency values such that the cancellation is generally more pronounced and the resulting WB (wide band) response is much smaller than in normal cases. Because the amplitude of the ABR shows a 10-fold range in the normal population, the response has to be properly nonnalized on the potential output of the cochlea, i.e., on the sum of the derived responses. Don (U.S. Pat. No. 6,264,616) presents a method in which the derived band responses are lined up such that wave V latencies overlap. This results in the “stacked ABR” and requires an expert to identify wave V in each derived band. Difficulties that may arise can be seen in FIG. 4a and 4b, where an untrained eye may not be able to determine wave V.

This invention provides a method which requires no expertise or detection of wave V to analyze the derived bands.

### SUMMARY OF THE INVENTION

An alternative to obtaining a stacked ABR is to normalize the power spectrum of the WB (wideband) on the SUM of the derived band power spectra.

There is therefore provided, according to an aspect of the invention, a method and apparatus of detecting abnormal auditory brainstem response. The apparatus comprises means for producing a broadband stimulus, electrodes for sensing an auditory brainstem response, and a processor connected to receive the auditory brainstem response and programmed to carry out the method. The method comprises the steps of receiving an acoustic response generated by applying a stimulus to an ear, the acoustic response comprising a set of frequencies, finding a power spectrum or equivalent transform for each of plural subsets of the set of frequencies, summing the power spectra or transform; and comparing the sum of the power spectra with the power spectrum or transform of the set of frequencies in the acoustic response. The subset of frequencies of the acoustic response may comprise the auditory brainstem response in a set of limited frequency ranges found by masking the acoustic response with white noise. According to a further aspect, the method is used to predict the existence of a tumor. According to a further aspect, the acoustic response is received by electrodes on an individual's forehead and mastoid. The acoustic response may be received differentially between an electrode on the high forehead and an electrode on the mastoid corresponding to the stimulated ear, and an electrode on the low forehead serves as a ground. According to a further aspect, the acoustic response of the cochlea is received. According to a further aspect, the sum of the plural subsets of the set of frequencies comprises a wide band response. According to a further aspect, the acoustic response is in the nonnal hearing range.

According to a further aspect of the invention, finding a subset of the set of frequencies comprises the steps of obtaining an umnasked acoustic response, obtaining masked acoustic responses by masking the stimulus with white noise in a frequency range, subtracting the masked acoustic response of the highest frequency range from the unmasked frequency response to obtain a subset of the set of frequencies, and subtracting the masked acoustic response from the next highest masked acoustic response for the remaining frequency ranges.

According to a further aspect of the invention comparing the sum of power spectra with the power spectrum of the set of frequencies in the acoustic response comprises normalizing the sum of power spectra to obtain a normalized sum and normalizing the power spectrum of the set of frequencies in the acoustic response to obtain a normalized reference and taking the ratio of the normalized sum and the normalized reference. A higher ratio of the normalized sum and the normalized reference may correspond to a higher probability of the existence of a tumor. The ratio of the normalized sum and the normalized reference may be compared to a ratio obtained from a group of people without abnormal auditory brainstem response or to a ratio obtained from the opposite ear of an individual. The peak in the ratio between 400-1000 Hz may be used as a predictor of the presence of a tumor. A processor may be used to predict the presence of a tumor.

These and other features of the invention will be apparent from the detailed description of the invention. The described method and apparatus may also be extended to apply to the detection of abnormalities in signals or responses from other bodies or parts of bodies, including human bodies, where phase information in sub-bands of the frequencies is lost in the wideband response.

### BRIEF DESCRIPTION OF THE DRAWINGS

There will now be given a brief description of the preferred embodiments of the invention, with reference to the drawings, by way of illustration only and not limiting the scope of the invention, in which like numerals refer to like elements, and in which:

FIG. 1 is a flow chart showing the steps involved in predicting a tumor;

FIG. 2 shows how a derived band is calculated;

FIG. 3 shows different derived acoustic responses of an ear to a stimulus;

FIG. 4 shows an apparatus for determining the acoustic response

FIG. 5 shows the power spectrum of the wideband response and the sum power spectrum.

FIG. 6 shows the ratio of the power spectrum of the wideband response and the sum power spectrum.

### DETAILED DESCRIPTION OF THE PREFERRED EMBODIMENTS

The word comprising is used in this document in its inclusive sense and does not exclude other features being present. The indefinite article “a” before an element specifies at least one of the elements is present, but does not exclude others of the same element being present. The term power spectrum refers to any power or magnitude calculated from a spectrum in which the phase information can be transformed and quantified, and may refer to various frequency domain (Fourier or Laplace transforms, complex demodulation), frequency-time domain (such as, but not restricted to, Wigner-, Choi-Williams-, and Rihacek-distributions) and “scaling”-time domain (various Wavelet transfonms) methods.

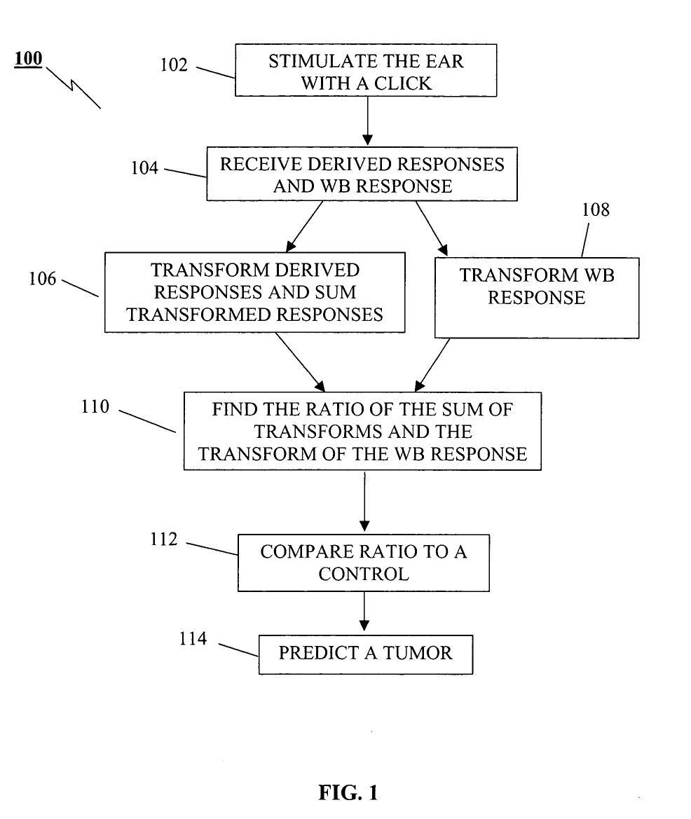

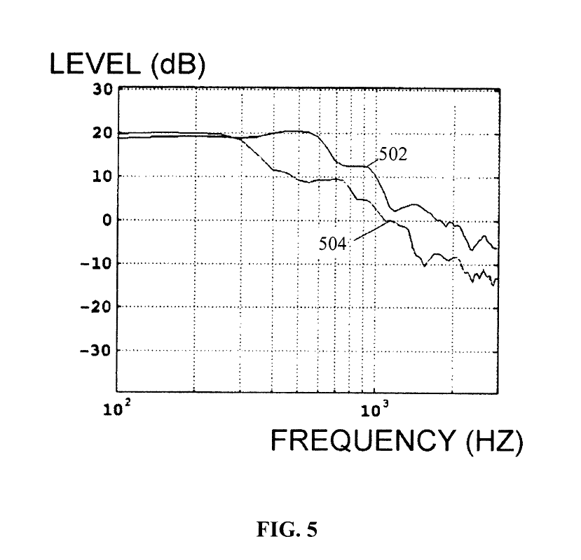

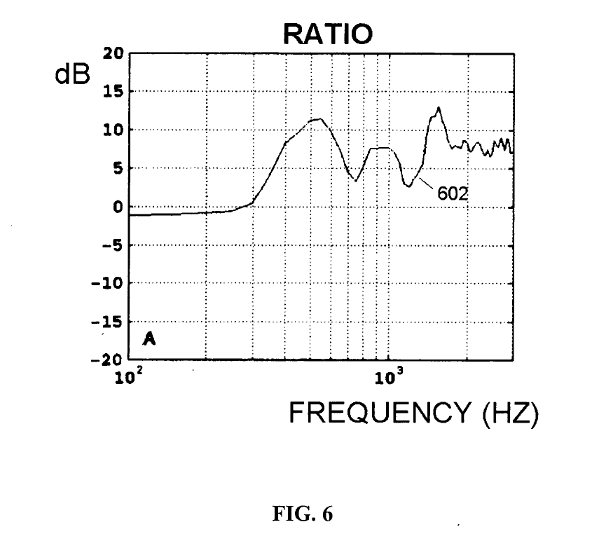

Referring to FIG. 1, there are shown steps in a method 100 of detecting an abnormal auditory brainstem response. The first step 102 is to apply a stimulus such as a click with a wide band frequency range to the ear of an individual. The auditory brainstem response (ABR), or acoustic response, is recorded from electrodes on the high forehead, the left and right mastoids and on the low forehead. The response to stimulation of the left ear is recorded differentially between the forehead, or any electrode close to the midline, and left mastoid electrode, and the response to stimulation of the right ear between the forehead, or any electrode close to the midline, and the right mastoid electrode. The low forehead electrode or any electrode in a convenient place serves as a ground. The signal is amplified to a convenient level, such as 100,000 times using a band pass filter setting between 100-3000 Hz. A continuous white noise level sufficient to just mask the ABR in response to clicks presented at, for instance, 60 dB nHL is used. The white noise is used to obtain the derived responses in 104, in addition to the WB (wideband), or unmasked response. The derived responses are subsets of frequencies in the original set of frequencies in the acoustic response. The original set of frequencies may be referred to as the WB (wideband) response. The method for obtaining the derived responses will be discussed below. In 106, the derived responses are transformed to find a power spectrum and are then summed to find a sum of power spectra of the derived responses. In 108, the WB response is transfonned to find a power spectrum of the WB response. The result is two power spectra that may be compared to see the effect of phase cancellation. By transforming the derived responses before summing, we are able to avoid the phase cancellation that occurs between the derived responses, while the WB response still contains that phase cancellation. The two normalized spectra can be seen in FIG. 5, where it can be seen that phase cancellation causes the level of the transformed WB response 504 is lower at higher frequencies than the sum of the transformed subset of frequencies. By comparing the two spectra, the effect of phase cancellation becomes apparent, and as this effect will be different in the presence of a tumor, it can be used as a diagnostic tool. As an example, if the ABR of two ears are compared, the ratio may be 12 for the left ear, and 8 for the right. In step 110 the transforms are compared by taking the ratio of the normalized sum of transforms and the normalized transform of the WB response. The plot of the ratio is shown in FIG. 6. The ratio is compared to a control in step 112, and the presence of a tumor is predicted in step 114 according to the results. The control may be the opposite ear of the patient, or a pre-determined average from a control group without abnormal ABR. In obtaining an average from a control group, it is necessary to divide the group according to age and gender, as these factors will also affect the latency of the cochlea, which is what generates the acoustic response that is measured.

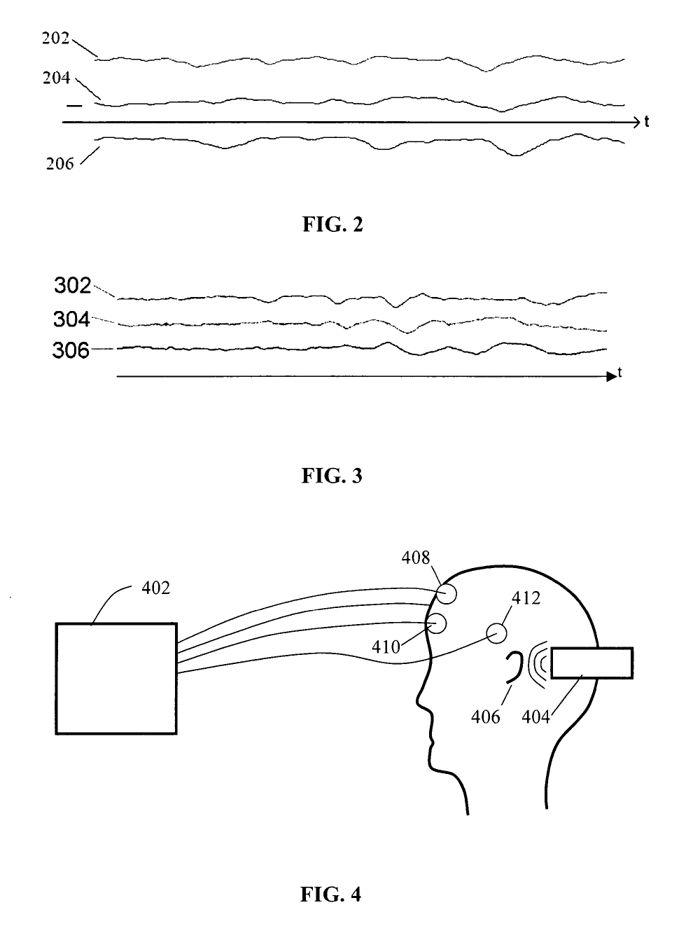

The method of obtaining derived acoustic responses as seen in FIG. 3 will now be discussed. FIG. 2 shows how this is done in the time domain for the highest frequency range, where an unmasked acoustic response 202 has been obtained. The stimulus is masked with white noise in a frequency range to obtain a masked response 204. The masked acoustic response is subtracted from the unmasked frequency response to obtain the response 206. For lower frequency ranges, the unmasked frequency range is replaced with the masked response of the next highest frequency range. For convenience, the set of frequencies chosen for the derived responses are selected to be in octaves, such as <500 Hz, 500-1000 Hz, 1000-2000 Hz, 2000-4000 Hz, 4000-8000 Hz, and >8000 Hz. Because of the mechanical response properties of the basilar membrane, high-pass masking does not affect frequency regions outside the pass band of the noise. Thus, the difference between the response to a click without noise and the response to a click in the presence of an 8 kHz high-pass noise would reflect the activity from that part of the cochlea that is masked by the 8 kHz high pass noise. Continuing, subtracting the response to a click in the presence of a 4 kHz high-pass noise from that recorded in the presence of a 8 kHz high-pass noise results in activity from the region in the cochlea that is masked by the 4 kHz high-pass noise but not by the 8 kHz high-pass noise. Continuing in this way one can derive the responses from octave wide regions along the cochlea. In FIG. 3, three examples of derived responses are shown, with 302 representing a higher frequency band than 304, and 304 representing a higher frequency band than 306. It can be seen that there are alternating positive-negative portions that tend to be out of phase, i.e., the responses cancel each other for specific latency ranges. As a consequence the ABR to a click tends to be smaller than the contributions from the individual octave bands would predict.

One observes that with decreasing high-pass cut-off frequency, and consequently greater masking of the normal hearing range (250-15,000 Hz), that the dominant ABR component present at 7-9 ms after stimulus onset is shifted to longer values. This reflects the masking of the high-frequency parts of the inner ear (cochlea) that cannot generate click-related activity. Because the response time (latency) of the high-frequency parts of the cochlea is shorter than those for the lower frequency components, a shift towards longer latencies occurs. In addition, the response amplitude may decrease somewhat.

The phase cancellation in the ABR as occurring in the time domain, can be quantified by comparing the sum of the power spectra of the derived responses with the power spectrum of the WB response to avoid response parts that would not contribute to the diagnosis such as the PAM (post-auricular muscle) and the stimulus artifact. By comparing the sum of the power spectra of the derived responses to the overall response with the power spectrum of the response to the click in the absence of any masking one observes that the SUM response is larger than the WB response. This difference reflects the degree of phase cancellation that occurs.

FIG. 6, line 602 quantifies this difference by showing the ratio of lines 502 and 504 (in fact the difference in dB of lines 502 and 504). There is no significant difference for frequencies below 300 Hz, whereas the difference above 1000 Hz may be affected mostly by the increase in noise resulting from the subtraction procedure to obtain the derived bands. We believe upon reasonable grounds that the dominant frequency components related to the identifiable peaks in the response would be in the 500-700 Hz region (period of 1.5-2 ms). This frequency region shows a prominent discrepancy in the ratio plot. It is believed on reasonable and probable grounds that the ratio (SUM/WB) will increase considerably when a tumor is present in the range of normal ratios or ratio x frequency (e.g., 300-750 Hz). Because there is generally only 1 peak in this range, the comparison may be automated such that a computer or other processor compares the value of the ratio in this range to a predetermined value or the value from the other ear, and provide a prediction, removing the need for a technician to analyze the data. A combination may also be used, where the ratio plot is displayed with the prediction, so that a visual check may be performed to ensure that the correct information was used in the prediction.

The apparatus that is used to carry out this method is shown in FIG. 4, where a device for generating a wideband stimulus or click stimulates the ear 406. Electrodes 408, 410, and 412 are used to receive signals generated by the patient, where signals are received differentially between 408 and 412, and the signals are sent to the processor 402. The processor is programmed to carry out the method as described. In using the term “processor is programmed”, it is understood that this encompasses any circuit capable of carrying out instructions, such as, but not limited to, a programmable microprocessor, a hard-wired circuit, or software that may be used by a computer. There may also be a combination of the above, for example, an amplifier and filter connected to a programmable microprocessor.

Those skilled in the art may make immaterial modifications to the invention described here without departing from the invention. The comparison may be carried out using the power-spectra or magnitude-spectra provided by the Fourier or Laplace transform or complex demodulation, of the averaged ABR and of each of the derived band ABRs. Using frequency-time domain and scaling-time domain representations the marginal frequency- and scaling-distributions are used to quantify these phase cancellation effects.

1. A method of detecting an abnormal response, the method comprising the steps of:
A) receiving a response generated by applying a stimulus to a body, the response comprising a complete set of frequencies;
B) combining transforms of each of plural subsets of the complete set of frequencies; and
C) comparing the combined transforms found in step B with a transform of the complete set of frequencies in the acoustic response.

2. The method of claim 1 in which the transform is a transform that results in a power spectrum of the frequencies contained in the response.

3. The method of claim 1 in which the response is an acoustic brainstem response generated by applying a stimulus to an ear.

4. The method of claim 2 in which combining transforms comprises the steps of:
B1) finding a transform of each of plural subsets of the set of frequencies; and
B2) summing the transforms found in step B1.

5. The method of claim 4 in which the transform is a transform that results in a power spectrum of the frequencies contained in the response.

6. The method of claim 5 in which the subset of frequencies of the acoustic response comprises the auditory brainstem response in a set of limited frequency ranges found by masking the acoustic response with white noise.

7. The method of claim 6 in which the subsets of the complete set of frequencies are found by the steps of:
obtaining an unmasked acoustic response;
obtaining masked acoustic responses by masking the stimulus with white noise in a frequency range;
subtracting the masked acoustic response of the highest frequency range from the
unmasked frequency response to obtain a subset of the set of frequencies; and
subtracting the masked acoustic response from the next highest masked acoustic response for the remaining frequency ranges.

8. The method of claim 3 in which the method is used to predict the existence of a tumor.

9. The method of claim 5 in which comparing the sum of power spectra with the power spectrum of the set of frequencies in the acoustic response comprises normalizing the sum of power spectra to obtain a normalized sum and normalizing the power spectrum of the set of frequencies in the acoustic response to obtain a normalized reference and taking the ratio of the normalized sum and the normalized reference.

10. The method of claim 9 in which a higher ratio of the normalized sum and the normalized reference corresponds to a higher probability of the existence of a tumor.

11. The method of claim 9 further comprising the step of comparing the ratio of the normalized sum and the normalized reference to a ratio obtained from a group of people without abnormal auditory brainstem response.

12. The method of claim 9 further comprising the step of comparing the ratio of the normalized sum and the normalized reference to a ratio obtained from the opposite ear of an individual.

13. The method of claim 9 in which a peak in the range between 500 to 700 Hz is used as a predictor of the presence of a tumor.

14. The method of claim 13 in which the acoustic response is received differentially between a first electrode on the mastoid corresponding to the stimulated ear and a second electrode.

15. The method of claim 1 in which a combination of the plural subsets of the complete set of frequencies comprises a wide band response.

16. An apparatus for detecting abnormal auditory brainstem response, the apparatus comprising:
means for producing a broadband stimulus;
electrodes for sensing an auditory brainstem response; and
a processor connected to receive the auditory brainstem response, the processor being programmed to:
A) receive an acoustic response generated by applying a stimulus to an ear, the acoustic response comprising a set of frequencies;
B) find a power spectrum for each of plural subsets of the set of frequencies;
C) sum the power spectra found in step B; and
D) compare the sum of the power spectra found in step C with the power spectrum of the set of frequencies in the acoustic response.

17. The apparatus of claim 16 in which the subset of frequencies of the acoustic response comprises the auditory brainstem response in a set of limited frequency ranges found by masking the acoustic response.

18. The apparatus of claim 17, in which the processor is further programmed to find the plural subsets of the set of frequencies by the steps of:
a) obtaining an unmasked acoustic response;
b) obtaining masked acoustic responses by masking the stimulus with white noise in a frequency range;
c) subtracting the masked acoustic response of the highest frequency range from the unmasked frequency response to obtain a subset of the set of frequencies; and
d) subtracting the masked acoustic response from the next highest masked acoustic response for the remaining frequency ranges.

19. The apparatus of claim 16 in which the processor is further programmed to predict the existence of a tumor from the result of step D.

20. Apparatus programmed to carry out the method of claim 1.
